(19) United States
(12) Patent Application Publication
(10) Pub. No.: US 2015/0289866 A1
(43) Pub. Date: Oct. 15, 2015

(54) Locking Device and Method of Use

(71) Applicant: Mark K. Bowen, Winnetka, IL (US)

(72) Inventors: Mark K. Bowen, Winnetka, IL (US); Xavier Marie Francois Cassard, Toulouse (FR)

(21) Appl. No.: 14/683,690

(22) Filed: Apr. 10, 2015

Related U.S. Application Data
(60) Provisional application No. 61/977,892, filed on Apr. 10, 2014.

(51) Int. Cl. A61B 17/04

(52) U.S. Cl. CPC A61B 17/0401; A61B 2017/0414; A61B 2017/0424; A61B 2017/0445; A61B 2017/0456; A61B 2017/0464; A61B 2017/045

(57) ABSTRACT

A locking device that includes an anchor base having a base outer wall and one or more base inner walls, wherein the one or more base inner walls form a base inner cavity. In addition, one or more cleats are provided that extend from the one or more base inner walls. Further, an anchor insert is provided having a first insert outer wall and a second insert outer wall, wherein the anchor insert is shaped to be received within the anchor base to provide securement of one or more sutures to the cleats.

### CROSS REFERENCE TO RELATED APPLICATION

This application claims the benefit of, and incorporates by reference, U.S. Provisional Patent Application No. 61/977,892 filed on Apr. 10, 2014 entitled “Suture Locking Device and Method of Use.”

### RELATED FIELD

The locking device and method of use relate to medical devices for use with repairing various living body portions.

### BACKGROUND

Anterior Cruciate Ligament (ACL) reconstruction using soft tissue grafts, among other things, can include various popular techniques to achieve functional knee stability for patients with damage to their knee joint, such as acute or chronic ACL tears. Unfortunately, many techniques for reconstruction, such as those utilizing a doubled semitendinosus and gracilis tendon, are not without issue, as various attempts to solve the challenge of a secure fixation of a soft tissue graft have produced numerous undesirable side effects, such as poor load-to-failure characteristics, stretching with cyclic loading, and functional instability.

### BRIEF SUMMARY

In at least some embodiments, the device and method of use relate to a locking device that includes an anchor base having a base outer wall and one or more base inner walls, wherein the one or more base inner walls form a base inner cavity, one or more cleats extending from the one or more base inner walls, and an anchor insert having a first insert outer wall and a second insert outer wall, wherein the anchor insert is shaped to be received within the anchor base to provide securement of one or more sutures to the cleats, when sutures are situated inside the base inner cavity.

In at least some other embodiments, the device and method of use relate to a locking device that includes a base having a base outer wall and an inner cavity, a first base inner wall joined to a second base inner wall at a first base corner and a second base corner to form the inner cavity, wherein the first base corner includes a first grip portion having one or more cleats, and the second base corner includes a second grip portion having one or more cleats, and an insert having a first insert outer wall and a second insert outer wall, wherein the first insert outer wall and second insert outer wall are joined by a first insert side wall and a second insert side wall, and wherein the anchor insert is shaped to be received at least partially within the base. In at least some embodiments, the locking device and method of use relate to a ligament reconstruction process that includes: a. securing a first suture to a first end of a tendon graft; b. securing a second suture to a second end of the tendon graft; c. implanting a first anchor base at least partially within an opening in a first bone; d. implanting a second anchor base at least partially within an opening in a second bone, and wherein the first and second bones are associated with a joint; e. passing the first suture through the opening in the first bone and the first anchor base; f. passing the second suture through the opening in the second bone and the second anchor base; g. inserting a first anchor insert into the first anchor base to abut the first suture into engagement with one or more cleats on one of the first anchor base and the first anchor insert to secure the first suture within the first anchor base; and h. inserting a second anchor insert into the second anchor base to abut the second suture into engagement with one or more cleats on one of the second anchor base and the second anchor insert to secure the second suture within the second anchor base.

In at least some embodiments, the locking device and method of use relate to a locking device that includes an anchor base having a base outer wall and one or more base inner walls, wherein the one or more base inner walls form a base inner cavity, a base bottom and a base top, wherein the base bottom and base top each include an opening therethrough, an anchor insert having a first insert outer wall and a second insert outer wall, wherein the anchor insert is shaped to be received within the anchor base, and one or more cleats extending from at least one of the one or more base inner walls, the first insert outer wall, and the second insert outer wall.

Other embodiments, aspects, and features of the device and method of use will be understood and appreciated upon a full reading of the detailed description and the claims that follow.

### BRIEF DESCRIPTION OF THE DRAWINGS

Embodiments of a locking device and method of use are disclosed with reference to the accompanying drawings, which are for illustrative purposes. The locking device and method of use is not limited in its application to the details of construction or the arrangement of the components illustrated in the drawings. The locking device and method of use is capable of other embodiments or of being practiced or carried out in other various ways. Like reference numerals are used to indicate like components. In the drawings:

FIG. 1 illustrates a frontal view of a knee joint with an exemplary embodiment of locking devices implanted in the knee joint to secure a graft to opposing bones of the joint;

FIG. 2A illustrates a front-top perspective view of an exemplary embodiment of an anchor base;

FIG. 2B illustrates a rear-bottom perspective view of the exemplary embodiment of the anchor base of FIG. 2A;

FIG. 3 illustrates a front view of the anchor base of FIG. 2;

FIG. 4 illustrates a top view of the anchor base of FIG. 2;

FIG. 5 illustrates a cross-sectional view of an inner wall of the anchor base of FIG. 2;

FIG. 6 illustrates a front perspective view of another exemplary embodiment of an anchor base;

FIG. 7 illustrates a top view of the anchor base of FIG. 6;

FIG. 8A illustrates a front-top perspective view of an exemplary embodiment of an anchor insert;

FIG. 8B illustrates a rear-bottom perspective view of the exemplary embodiment of the anchor insert of FIG. 8A;

FIG. 9 illustrates a front view of the anchor insert of FIG. 8;

FIG. 10 illustrates a top view of the anchor insert of FIG. 8;

FIG. 11 illustrates a front perspective view of another embodiment of an anchor insert;

FIG. 12 illustrates a front view of the anchor insert of FIG. 8 prior to installation in the anchor base of FIG. 2, along with sutures passing therethrough;

FIG. 13 illustrates an upper cross-sectional view of the anchor base of FIG. 12;

FIG. 14 illustrates a middle cross-sectional view of the anchor base of FIG. 12;

FIG. 15 illustrates a lower cross-sectional view of the anchor base of FIG. 12;

FIG. 16 illustrates a front view of the anchor insert of FIG. 12 situated in the anchor base of FIG. 12, with the sutures secured therein;

FIG. 17 illustrates an upper cross-sectional view of FIG. 16;

FIG. 18 illustrates a middle cross-sectional view of the anchor base of FIG. 16;

FIG. 19 illustrates a lower cross-sectional view of the anchor base of FIG. 16;

FIG. 20 illustrates a lateral view of a knee joint with locking devices implanted in the knee joint to secure a graft to opposing bones of the joint;

FIG. 21 illustrates a front-top perspective view of another exemplary embodiment of an anchor base;

FIG. 22 illustrates a rear-bottom perspective view of the exemplary embodiment of the anchor base of FIG. 21;

FIG. 23 illustrates a front-top perspective view of another exemplary embodiment of an anchor insert; and

FIG. 24 illustrates a rear-bottom perspective view of the exemplary embodiment of the anchor insert of FIG. 23.

### DETAILED DESCRIPTION

### Definitions

The term “anchor base”, also referenced as an “implant base” and “base,” means a structure configured to be implanted into the cartilage or bone of a living body and to receive and secure one or more sutures.

The term “anchor insert,” also referenced as an “implant insert” and “insert,” means a structure configured to be inserted into the anchor base to assist with securement of one or more sutures.

The term “suture” means one or more fibers or strands suitable for insertion in a living body and for tethering a graph to the locking device.

The terms “comprising,” “including,” “having,” and their derivatives, are not intended to exclude the presence of any additional component, step or procedure, whether or not the same is specifically disclosed.

### DESCRIPTION

As described herein, an exemplary locking device is provided that includes an anchor base that is suitable for securement in an exemplary body portion, such as a bone, and an anchor insert for cooperatively securing exemplary fasteners, such as sutures, within the anchor base. In at least some embodiments, the sutures are secured to opposite ends of a graft and then to respective locking devices that are each implanted in separate bones.

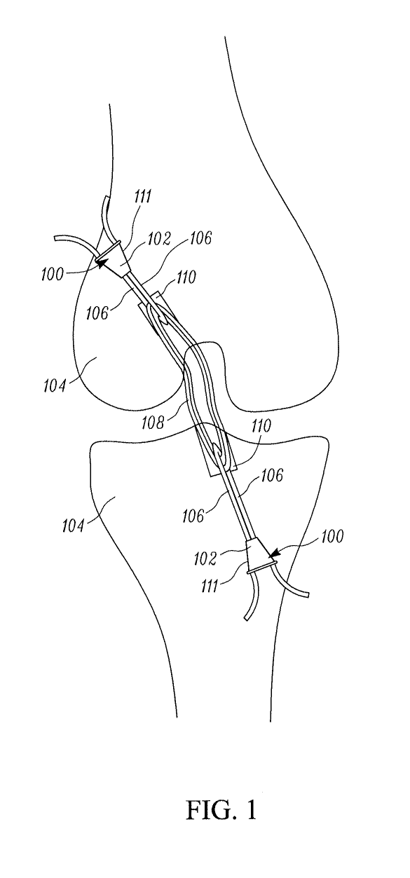

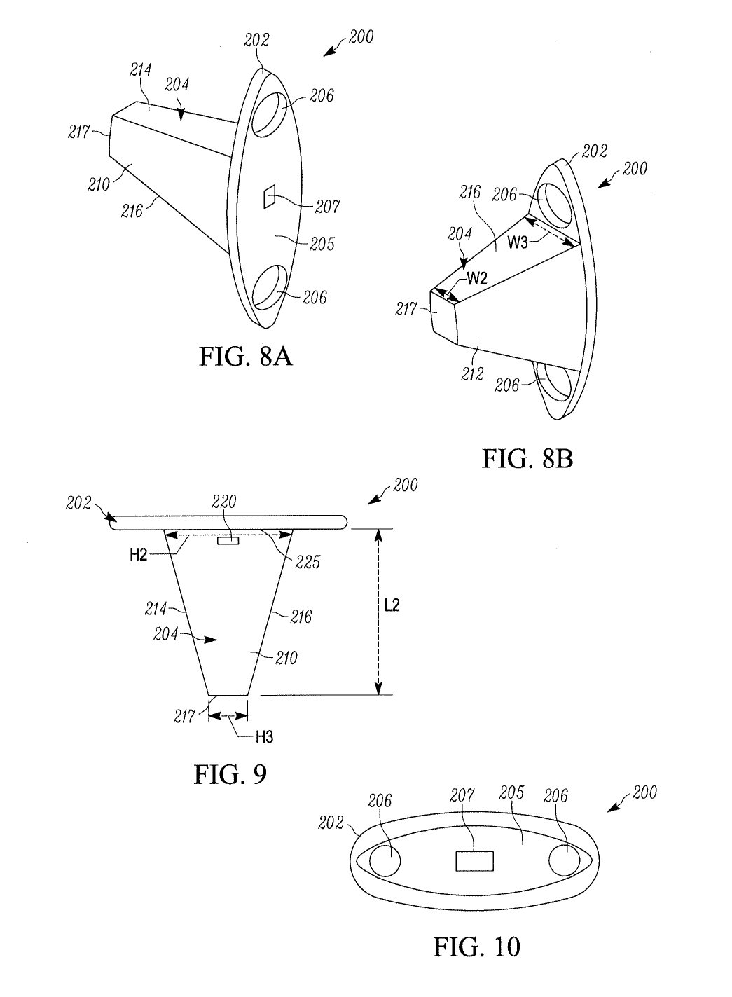

Referring to FIG. 1, an exemplary embodiment of a pair of locking devices 100 is shown implanted in the bones adjacent a knee joint. Each locking device 100 includes an anchor base 102, suitable for securing to an exemplary body portion, such as a bone 104 (e.g., a femur, tibia, etc.), and an anchor insert 200 (see FIG. 8) for insertion into the anchor base 102 to assist with securing exemplary sutures 106 to the anchor base 102. As shown, in at least some embodiments, the sutures 106 are secured to the implanted locking device 100 at one end and engaged with a portion of an exemplary graft 108 at the other end. In this manner, the graft 108 can be tethered at one end to a bone with a first locking device and tethered at the other end to another bone by a second locking device. Such a use provides securement of a graft to each bone that forms a joint. The graft 108 can include any one of numerous types of materials suitable for use in a living body, such as a natural or artificial tendon, and other soft tissue graft materials, etc.

A pair of locking devices is shown in FIG. 1 for use in the knee joint, although it is not necessary for a pair of locking devices 100 to be used in all applications. For example, in the case where a tendon or other portion of a living body remains naturally secured or repairably secured to a bone, a single locking device 100 can be used to tether an unattached end of the tendon or other body portion to a desired bone. In at least some embodiments, one or more passages 110 can be artificially formed in the bone 104 to allow the passage of the graft 108 and sutures 106 therethrough. Further, a socket 111 can be artificially formed in the bone, such as by drilling, or machining with a bone rasping tool to allow for implantation of the anchor base 102. In at least some embodiments, materials not considered sutures can be secured inside the locking device 100.

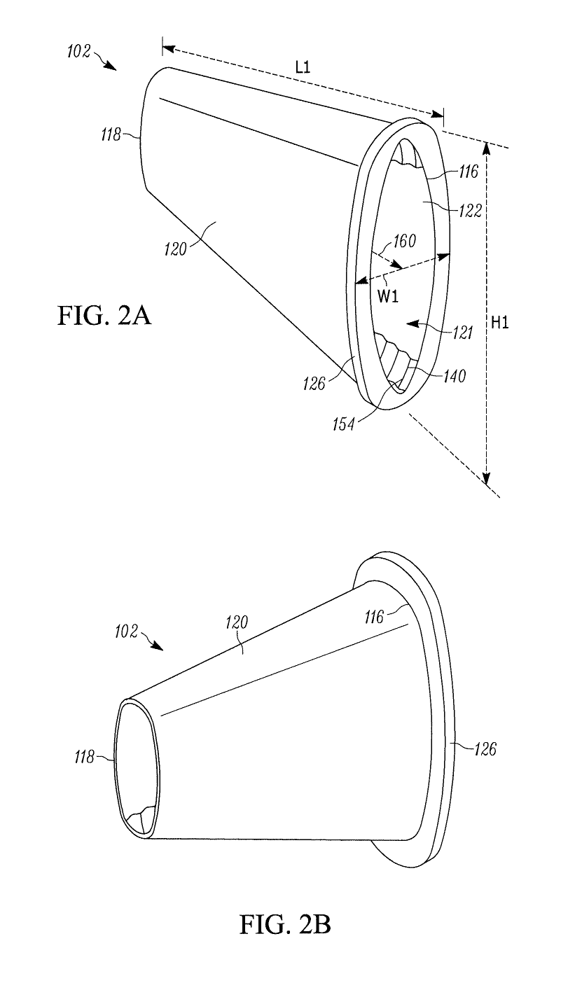

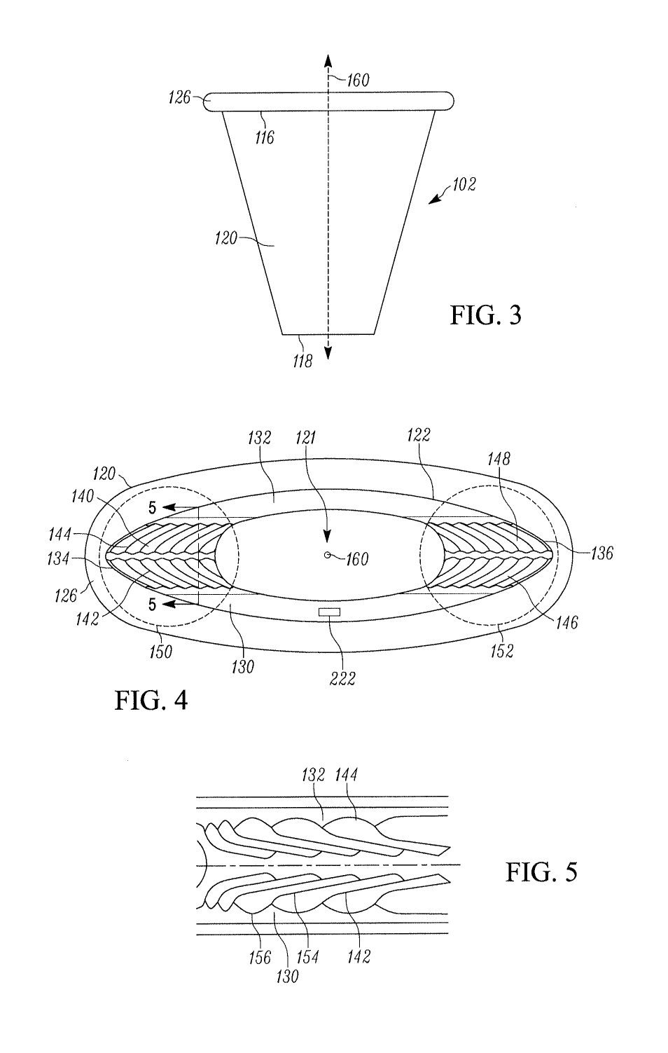

Referring to FIGS. 2A, 2B, 3 and 4, a front-top perspective view of an exemplary embodiment of the anchor base 102 is provided, along with a rear-bottom perspective view, a front view and a top view, respectively. The anchor base 102 has a base top 116, a base bottom 118, and a base outer wall 120. In at least some embodiments, the base top 116 and base bottom 118 are at least partially open to allow sutures 106 to pass through the anchor base 102. In addition, the anchor base 102 includes a base inner cavity 121 formed by one or more base inner walls 122. In at least some embodiments, the anchor base 102 includes a lip 126 that extends radially (e.g., at about a 90 degree angle relative to the longitudinal center axis 160) from the base outer wall 120 and around to the base inner wall 122, as shown. In at least some other embodiments the lip 126 can extend along one of various other angles relative to the longitudinal center axis 160 to accommodate implantation flush to the surface of the bone 104. The lip 126 can serve several functions. For example, the lip 126 can provide structural rigidity to the anchor base 102 and in at least some embodiments, provide a stop to prevent the anchor base 102 from receding into the bone either during implantation or post implantation. Further, the lip 126 can serve to reduce contact of the sutures 106 with the bone, as such, the sutures 106 are less likely to degrade or be degraded by the bone adjacent the base top 116.

The anchor base 102 can be comprised of various different shapes both outside along the base outer wall 120 and inside along the base inner wall 122. The length L1, width W1, and height H1 can all vary to accommodate a desired implementation. For example, for implantation into a larger diameter bone, such as a femur, the length L1 can be greater than the height H1, whereas in a small diameter bone, such as a clavicle, the length L1 can be reduced to accommodate the reduced diameter without risking fracture of the bone during or after implantation. Likewise, the height H1 and width W1 can vary to accommodate the size of the bone where the anchor base 102 is to be implanted. In addition, the height H1, width W1, or length L1 of the anchor base 102 can be chosen to provide the required strength necessary to sufficiently anchor a graft to the bone based on the expected load to the sutures 106 for the joint being repaired. In at least some embodiments, the anchor base 102 is elongated having a generally oval cross-section, although in other embodiments, the anchor base 102 can be comprised of other shapes and dimensions, such as four-leaf clover, star-shaped, tubular, etc. Further, in at least some embodiments, the anchor base 102 has a length L1 between about 8 millimeters and about 20 millimeters, a height H1 between about 6 millimeters and about 15 millimeters and a width W1 between about 4 millimeters and about 10 millimeters, while still in other embodiments, the height H1, length L1, and width W1 can be greater than or less than the aforementioned dimensions. Further, in at least some embodiments, the anchor base 102 tapers as it extends from the base top 116 towards the base bottom 118.

The base inner cavity 121 can vary in size and shape to accommodate the base outer wall 120, as well as to accommodate various sizes and shapes of the anchor insert 200 and various types and sizes of sutures 106. As noted above, the base inner cavity 121 is formed by one or more base inner walls 122. In at least some embodiments, the base inner cavity 121 is formed from a single base inner wall 122 that extends around the base inner cavity 121 opposite the base outer wall 120. In at least some other embodiments, the base inner wall 122 is comprised of a first base inner wall 130 situated opposite a second base inner wall 132, with the first base inner wall 130 and second base inner wall 132 joined by a first base corner 134 and a second base corner 136 to form the base inner cavity 121. In at least some other embodiments, the first base corner 134 and second base corner 136 each form a wedge shape to assist with securement of the sutures 106. In at least some other embodiments, the base inner wall 122 is comprised of several interconnected walls that form various configurations inside the base inner cavity 121 for securing the sutures 106.

Referring to FIGS. 4 and 5, one or more cleats 140 are situated inside the base inner cavity 121. In at least some embodiments, the one or more cleats 140 are situated along the base inner wall 122. In at least some other embodiments, the one or more cleats 140 include a column of first cleats 142 situated opposite a column of second cleats 144, with the first cleats 142 and second cleats 144 extending at least partially along a length of the first base corner 134, and a column of third cleats 146 situated opposite a column of fourth cleats 148, with the third cleats 146 and fourth cleats 148 extending at least partially along a length of the second base corner 136, and wherein the first base corner 134 in combination with the first cleats 142 and second cleats 144 forms a first grip portion 150 and the second base corner 136 in combination with the third cleats 146 and fourth cleats 148, forms a second grip portion 152. In addition, the anchor base 102 may include only a single grip portion or more than two grip portions inside the base inner cavity 121 with each grip portion including one or more cleats 140. In at least some embodiments, the first grip portion 150 and second grip portion 152 can be comprised of less or more cleats 140, and situated in various other positions and configurations along the one or more base inner walls 122.

The cleats 140 can vary in size and shape, although in at least some embodiments, they include alternating ridges 154 and troughs 156 (see FIG. 5) that are integrally formed with or secured to the one or more base inner walls 122. In addition to the cleats 140 as shown and described, in at least some embodiments, the cleats 140 can include various other shapes, sizes, and styles of protrusions to form the first grip portion 150 and second grip portion 152, such that it is understood that the term “cleat” is not to be limited to a shape having a ridge and trough, but can include, for example, one or more planar walls protruding from the base inner walls 122 such that an edge is provided to engage the sutures 106. The cleats 140 can be situated at various angles relative to a base longitudinal axis 160 extending centrally through the anchor base 102. For example, in at least some embodiments, the cleats 140 are angled to point at least partially downwards as they extend down the length of the one or more base inner walls 122 and away from the base longitudinal axis 160 such that the ridges 154 can engage an inserted suture 106.

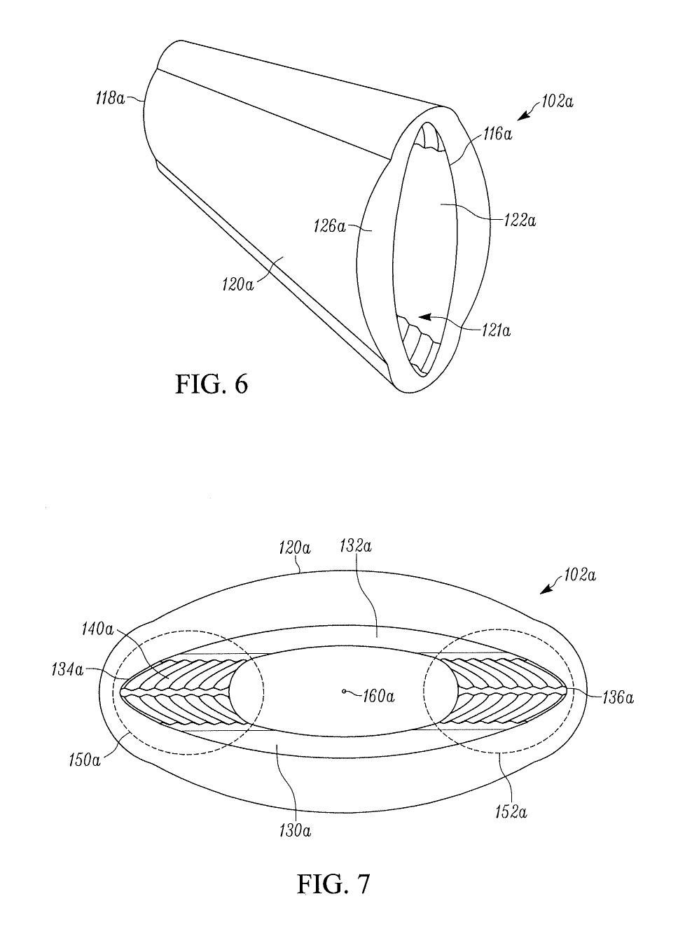

Referring to FIGS. 6 and 7, a front perspective view of another exemplary embodiment of the anchor base 102, is provided, along with a top view. As discussed above, the anchor base can vary in size in shape, both along the base outer wall and base inner cavity. The anchor base in FIGS. 6 and 7 has substantially the same components as the anchor base 102 described above, as such FIGS. 6 and 7 have the same elements as described above, but they can be varied in shape and size, and therefore are identified with an “a” after their numbers (e.g., 102a) and it is to be understood that the anchor base 102a and its sub-elements provide similar function to the anchor base 102 described above.

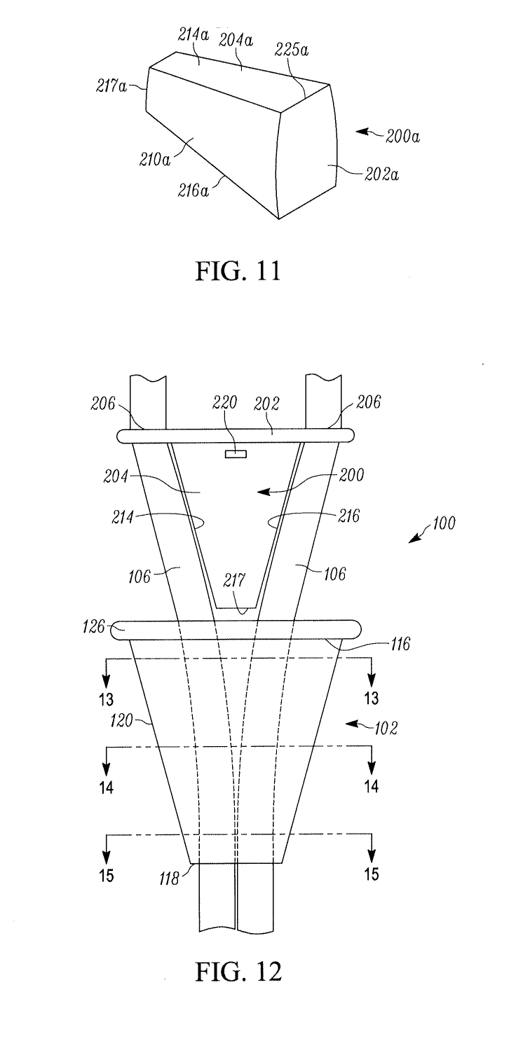

Referring now to FIGS. 8A, 8B, 9, and 10, a front-top perspective view of an exemplary embodiment of the anchor insert 200 is provided, along with a rear-bottom perspective view, a front view, and a top view, respectively. The anchor base 102 is sized and shaped to receive the anchor insert 200, which includes an insert top portion 202 and an insert wedge portion 204 that extends downward from the insert top portion 202. In at least some embodiments, the insert top portion 202 is planar, and extends beyond the extents of the walls of the insert wedge portion 204, and includes one or more apertures 206 for one or more sutures to be threaded therethrough. The insert top portion 202 further includes an upper surface 205 that in at least some embodiments, includes an indent 207 or protrusion for engagement with a impacting tool to assist with insertion of the anchor insert 200 into the anchor base 102. In at least some other embodiments, such as shown in FIG. 11, an insert top portion 202a may not extend beyond the extents of an insert wedge portion 204a and therefore may not include one or more apertures.

In at least some embodiments, the shape of the insert top portion 202 is complimentary to the shape of the one or more anchor base inner walls 122 to allow the insert top portion 202 to sit flush with or to recede into the base top 116. The one or more apertures 206 allow the sutures 106 to pass through the anchor insert 200 when installed in the anchor base 102 in a covering manner. In at least some embodiments, the insert wedge portion 204 includes an first insert outer wall 210 and an second insert outer wall 212, wherein the first insert outer wall 210 and second insert outer wall 212 are joined by a first insert side wall 214 and a second insert side wall 216. In at least some embodiments, the insert wedge portion 204 tapers down as it extends from an insert wedge top 225 to an insert bottom 217. In at least some embodiments, the first insert outer wall 210 and second insert outer wall 212 have the same shape and dimension, as do the first insert side wall 214 and the second insert side wall 216. In other embodiments, the walls 210, 212, 214, 216 can all vary in size and shape as desired, and can be joined by a greater or lesser quantity of walls to form the insert wedge portion 204. In at least some embodiments, the first insert outer wall 210 and second insert outer wall 212 are planar, while in some other embodiments, they are bowed inwards or outwards to be matingly received by the base inner cavity 121. More particularly, the first insert outer wall 210 and second insert outer wall 212 can be generally contoured to match the first base inner wall 130 and second base inner wall 132. In this manner, the anchor insert 200 can be securely engaged with the anchor base 102. Engagement of the anchor insert 200 with the anchor base 102 can be via fitment, such as through a frictional engagement, or through a locking mechanism, such as one or more engageable tabs 220 that extend from one or more of the walls of the anchor insert 200 for reception by one or more recesses 222 (see FIG. 4) in the anchor base inner wall 122, or vice-versa. The first insert side wall 214 and the second insert side wall 216 can vary in size and shape, such as planar, or bowed inwards or outwards to accommodate and engage the sutures 106 and in some cases, the cleats 140. Further, in at least some embodiments, the insert wedge portion 204 has a length L2 between about 6 millimeters and about 20 millimeters, a height H2 at the insert wedge top 225 of about 4 millimeters to about 15 millimeters, a height H3 at an insert bottom 217 between about 2 millimeters and about 10 millimeters, a width W2 at an insert wedge top 225 between about 4 millimeters and about 10 millimeters, a width W3 at an insert bottom 217 between about 2 millimeters and about 6 millimeters, while still in other embodiments, the heights H2, H3, length L2, and widths W2, W3 can be greater than or less than the aforementioned dimensions.

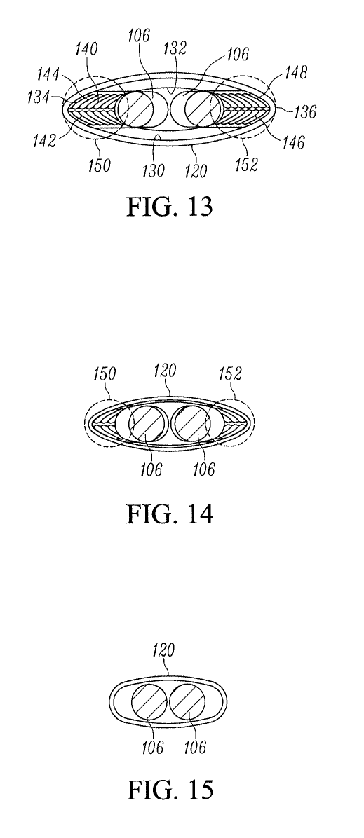

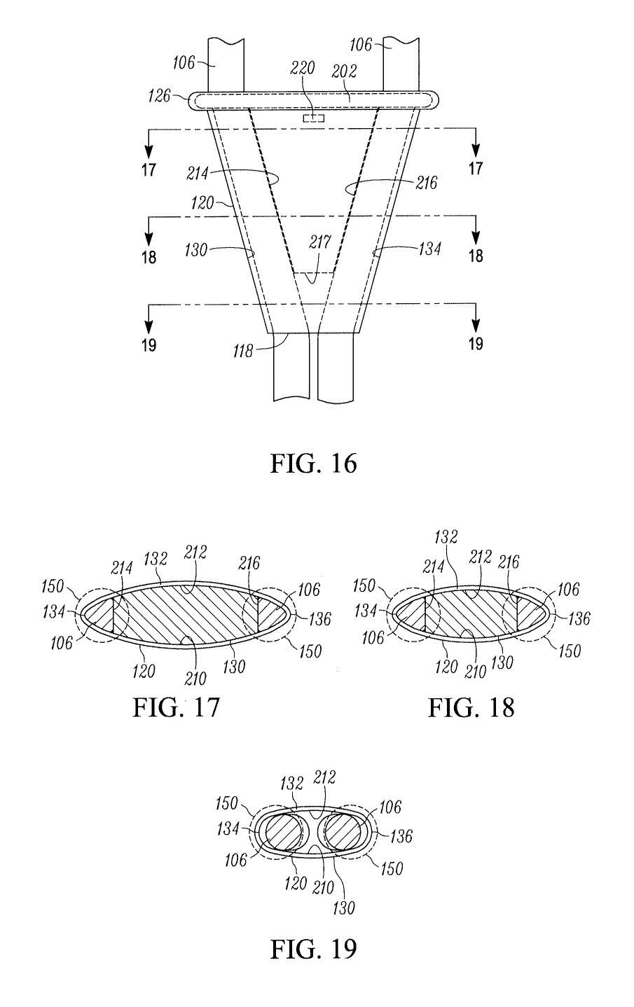

Referring to FIG. 12, a front view of the locking device 100 is shown that includes the anchor insert 200 prior to insertion into the anchor base 102. In addition, sutures 106 are shown passing through the locking device 100. In addition, FIGS. 13-15 provide cross-sectional views of the anchor base 102 with the sutures 106 therein. FIG. 16 is another front view of the locking device 100 shown in FIG. 12, which includes the anchor base 102 after the anchor insert 200 has been inserted therein to secure the sutures 106 into the anchor base 102. In addition, FIGS. 17-19 provide cross-sectional views of the locking device 100 with the sutures 106 secured therein. It is to be noted that reference herein to the term “sutures” can include either separate sutures (not attached to each other), or a first and second end of a single suture (such as a suture that has been looped around a graft).

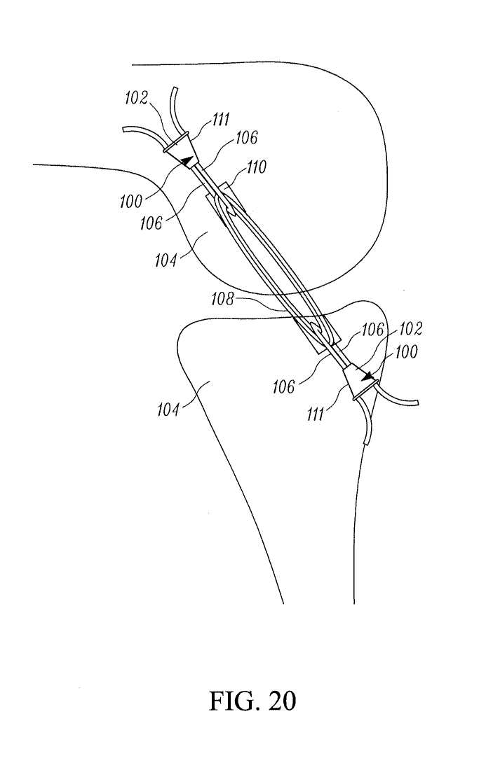

With further reference to FIGS. 12-19, in at least some embodiments, the anchor base 102 is implanted into the bone 104, this can be accomplished using one of many securement procedures, such as drilling the bone 104 to form a socket 111 (FIGS. 1 and 20) and impacting the anchor base 102 into the socket 111. In at least some embodiments, the anchor base 102 includes texturing or other protrusions along its outer wall 120 to assist with initial and post-operative securement to the bone 104. In at least some embodiments, the protrusions along the outer wall 120 can include spiral threads to allow the anchor base 102 to be screwed into the bone 104. Additional securement procedures can be performed, such as adding adhesive or other materials to the implant socket, where the materials can serve to promote regrowth of bone to engage the anchor base 102. After implantation of the anchor base 102, one or more sutures 106 are passed through the base bottom 118 and base top 116, and the one or more sutures 106 are fed through the one or more apertures 206 of the anchor insert 200 (if the anchor insert includes apertures 206), then the one or more sutures are positioned to abut the cleats 140 in at least one of the first and second grip portions 150, 152. As the anchor insert 200 is inserted into the anchor base 102, the first insert side wall 214 pushes the suture 106 into the cleats 140 in the first grip portion 150 and maintains pressure on the suture 106 to keep the suture 106 from sliding out of the anchor base 102. Likewise, the second insert side wall 216 pushes an opposite end of the suture 106 (or a different suture, if included) into the cleats 140 in the second grip portion 152, to maintain pressure on the suture and keep it from sliding out of the anchor base 102.

The locking device 100 can be used to secure (i.e., fixate) sutures in bone in a variety of medical procedures, for example, quadrupled single tendon knee ligament reconstruction techniques for both ACL and PCL (posterior cruciate ligament) tears, as well as various other techniques, such as repairs of acromioclavicular joint dislocations, and ankle ligament reconstructions.

At least one method of use of the locking device 100 includes the quadrupled single tendon technique for ACL repair, as described below and shown in FIGS. 1 and 20. The method of repair described below is intended to be an exemplary use of the locking device 100, and it is to be understood that the securement of sutures 106 to the locking device 100 and the securement of the locking device 100 to the bone 104 can be modified as necessary to accommodate installation in other bones for the repair of other body parts.

Quadrupled single tendon techniques have been shown to provide a lower morbidity with less tendon sacrifice compared to a doubled tendon technique. They can be performed with all-inside techniques requiring less bone removal. The single hamstring graft is quadrupled and suspended at each end by wrapping it around a looped strand of soft, inelastic braided suture. This technique also provides for tight press-fit and circumferential contact of the graft in bone sockets. Fixation of the graft in the Femur and Tibia can be achieved using the locking device that is implanted in the bone and rigidly locks one or more sutures. Fixation of the suture is close to the graft to minimize any “bungee-cord” effect and does not touch the graft itself.

Careful positioning of an ACL graft centrally within a native ACL footprint anatomy appears to be critical to the success of reconstruction of an ACL. Placements of bone tunnels or sockets in the femur have traditionally been performed using outside-in, trans-tibial or medial portal techniques. In at least some embodiments, the ACL reconstruction technique described herein capitalizes on the accurate, reproducible, and tibia-independent characteristics of an outside-in femoral approach to the ACL footprint. The socket is atraumatically retro-reamed and is achieved using a cosmetic, percutaneous approach. The tibial socket is prepared independent of any conditions of the femoral socket and can be placed accurately in the center of the tibial footprint.

Performance of an exemplary quadrupled single tendon technique for ACL repair using the locking device 100, as well as providing some general instruction for securing sutures to the locking device itself, is provided below. Although numerous details have been provided for performing this procedure, it is to be understood that various details have been omitted, such as anesthetization, etc. In addition, it is understood that a person skilled in the art would understand various references to the parts and uses of tools well known in the art, such as surgical drills and drill guide systems, retroreamers, etc. Further, the numerical designations provided below are exemplary, and therefore, may increase or decrease as needed to adjust for numerous variables such as the size of the bone, the graft, body dimensions, etc.

Exemplary Patient Positioning

The patient is positioned on the table with the knee at 90 degrees with the foot on a padded transverse post and a thigh post high on the lateral leg because the majority of the procedure including graft harvest, notch preparation, guide pin placement, femoral socket retro-reaming and tibial tunnel drilling is performed in this position. A tourniquet is placed high on the thigh to allow maximum length of the hamstring and is kept inflated only during the approach and harvest.

Exemplary Portal Placement

In at least some cases, proper portal placement can be critical to the success of ACL reconstruction. Two strategically placed portals can augment visualization of the ACL footprint and accuracy of the reconstruction.
A standard anterolateral (AL) viewing portal provides visualization throughout the procedure and accurate placement of the anteromedial (AM) portal. This portal is created high and close to the patella tendon.
A standard anteromedial (AM) portal is used for meniscal surgery, instrumentation, and femoral and tibial guide placement. This is created under direct visualization after testing the position with an 18-gauge needle. It should be close to the patella tendon, near the inferior pole of the patella and enter the joint out of the fat pad and safely above the medial meniscus. The portal is enlarged late in the procedure to allow for passage of the graft into the knee.

Exemplary Graft Harvest

To harvest the Semitendinosus tendon, make an oblique incision over the pes anserinus, which can be palpated approximately three finger breadths below the top of the medial tibial plateau. Carefully identify the Sartorius fascia, re-palpate the tendons and open the fascia on the cephalad side of the tendons. Retract the fascia and observe the tendons from the deep side (underneath) of the fascia. Palpate and hook the inferior most, larger, Semitendinosus tendon with a right angle clamp and pass a penrose drain around it to allow for retraction. Carefully remove soft tissue around the tendon and incise the fascial attachments to the medial gastrocnemius muscle. Use the open-ended tendon stripper to remove the tendon from its proximal muscle and gain as much tendon length as possible. Next, strip the remaining muscle fibers from the proximal hamstring using the side of a metzenbaum scissors. Finally, pull the tendon distally and incise from its attachment to the gracilis and release off the tibia to maximize length.

Exemplary Graft Preparation

The curved uprights of the Graft Preparation Station are set at a distance proportional to the height of the patient, in at least some embodiments the following settings can be used:
5′ 6″ or shorter 50 millimeter
5′6″ to 5′10″ 55 millimeter
5′10″ or taller 60 millimeter
   The harvested semitendinosus is wrapped around the curved uprights on the Preparation Station to make a full graft thickness of four strands. The side with a six thickness of graft is then sewn together by placing sutures across all strands and back again, tying a knot, wrapping one strand around the whole graft and tying four more knots. The suture is placed approximately 15 millimeters from the end of the graft to mark the distance that will be fully buried in the tibial tunnel. The uprights are then manually spread apart to equalize tension in the limbs of the graft. Two or three further sutures are now placed between the initial stitch and the 2 millimeter fixation suture on the six tendon side that is intended for the tibial socket. A single suture is then added at approximately 10 millimeters from the end on the closed loop end of the graft to compress the strands together and mark the expected length of graft that will enter the socket. Each of these sutures is intended to pass back and forth through all strands of the graft, a knot is tied and one limb is wrapped around the graft and four further knots are added. Finally the graft is further tensioned to approximately 200 N (Newtons) for one minute and both ends are sized.

Exemplary Notch Preparation

Once the Hamstring graft has been harvested, prepared and sized, position the knee at 90 degrees. Perform a standard arthroscopic evaluation of the knee, including evaluation of the ACL, meniscal, and articular cartilage pathology. If needed, a limited notchplasty can be utilized to allow evaluation of the femoral footprint attachment of the ACL. A 5.5 millimeter aggressive bone cutter shaver can be highly efficient for removal of soft tissue and bone. Remnants of the femoral and tibial footprints are preserved until guide pins are placed, to assure accurate recreation of the ACL anatomy.

Exemplary Guide Pin Placement—Femur:

1. While viewing with a standard 30 degrees arthroscope in the lateral portal, position the tip of the Medial Portal Guide at the center of the femoral footprint between the Anteromedial (AM) and Posterolateral (PL) bundles. Outside the knee, the guide is angled approximately 45 degrees in both the coronal and sagittal planes. Care is taken to not angle too low to the lateral femur to avoid inferior entrance and possible compromised fixation.

2. Once the guide is in position, at the point where the bullet of the guide is projected to enter the lateral thigh skin, a #11 blade can be used to make a small stab (approximately 1 centimeter) incision through the skin and the illiotibial band.

3. Insert the bullet through the guide and the slide the bullet down to the lateral femur. Based on the angle of the guide discussed above the guide pin should be directed toward the flare of the lateral femur.

4. While maintaining intra-articular visualization of the guide tip, advance a 2.4 millimeter drill tip guide pin into the femoral footprint. If repositioning is necessary, move the tip of the guide with the first guide pin left in place but withdrawn so it is just visible, and drill a second pin into the desired site.

Exemplary Guide Pin Placement—Tibia:

1. Identify the center of the anatomic ACL insertion site on the tibial plateau.

2. Set a drill guide at a 60° angle and insert it through the AM portal into the knee joint. Place the tip of the aimer at the center of the tibial footprint of the ACL. Direct the bullet down to the tibial surface utilizing the incision made for the graft harvest. The desired total tunnel length is approximately 40 to 45 millimeters to accommodate 15 to 20 millimeters of graft in the socket and the 20 millimeter locking device

3. Drill a 2.4 millimeter guide pin into the tibial footprint.

Exemplary Socket Preparation:

1. Once the guide pins are satisfactorily positioned, the sockets are prepared using identical methods except that the intended tibial socket depth is 15 millimeters and the femoral depth is chosen to be 10 to 15 millimeters depending on available graft length.

2. Over-drill the 2.4 millimeter guide pin with the 4.5 millimeter drill bit.

3. Keeping the 2.4 millimeter guide wire in place, remove the 4.5 drill bit and introduce the appropriate size fixed wing retroreamer over the guide wire. Select a reamer that corresponds with the measured size of the appropriate side of the hamstring graft. It is important to orient the wings of the retroreamer perpendicular to the long axis of the femur and tibia as this will later determine the orientation of the locking device.

4. Retro-ream the socket until it reaches the intended depth. Remove the Retroreamer leaving the 2.4 millimeter guide pin in place.

5. Place the cortical broach/cannula and its insertion guide over the wire and on the femoral side guide it through the transverse cut in the fascia, down to the cortex. The tibial side preparation is easily visualized due to the incision for the graft harvest. As with the retroreamer, orient the broach perpendicular to the long axis of the femur and transverse on the tibia. Impact the broach until the collar stops at the cortex and then remove the insertion guide and plug the cannula to prevent water egress.

6. Debride the edges of the tunnel to smooth out the entrance and remove all bone debris that might fall behind the lateral femoral condyle using a 5.5 millimeter shaver.

Exemplary Graft Passage:

1. Once both sockets have been prepared, the AM portal is enlarged to accommodate passage of the graft. Introduce a double opening “Suture-shuttle” through both the tibial and femoral cannulae and retrieve it through the medial portal. It is advantageous to make a clean passage of the arthroscopic grasper when retrieving each “Suture-shuttle” so fat pat or soft tissue does not interfere with free graft passage into the knee.

2. Starting with the femur, use the two openings in the “Suture-shuttle” to pass each end of the grafts sutures intended for the femur through the medial portal, the notch and out through the lateral incision. The sutures are spread and one side colored with a skin marker to keep them separated and avoid twisting them on each other.

3. The graft sutures are grasped with a straight hemostat that is applied directly at the end of the cannula in the slot provided. The hemostat is then turned against the base of the cannula, wrapping the sutures around it as it rotates. This powerfully pulls the graft into the socket allowing it to be press fit. It is critical to hold and stabilize the cannula firmly with one hand while rotating the hemostat with the other. The fit and security of the fixation may be compromised if the cannula wobbles during the graft docking.

4. Once the graft is docked in the femur, the graft sutures from the tibial side of the graft are passed through the two openings in the “Suture-shuttle” which is then pulled down through the cannula, thus pulling the graft through the medial portal and the graft sutures down the tibial socket and out the tibia. In the same manner, the graft is press-fit into the socket by grasping the graft sutures with a hemostat and turning against the slotted end of the cannula.

5. Accurate placement of the graft ends in each socket is now confirmed, noting the disappearance of the marking sutures on both the femoral and tibial sides of the graft. The amount of graft in each socket can be adjusted by further turning of the hemostat against the end of the cannulae. The graft should now be fully tensioned in the knee.

Exemplary Graft Fixation—Femur:

1. The graft is fixed in the femur first. The cannula is removed using the slotted removal hammer with care to keep the orientation so the bone is not damaged. It is also important to maintain the orientation of the sutures so that they do not twist on each other.

2. The anchor base 102 of the locking device 100, secured on a driver, is then loaded with the graft sutures, while maintaining their orientation to avoid twisting. The anchor base 102 is advanced to the bone 104 and impacted in place, keeping tension on the sutures to prevent bunching up or binding.

3. The sutures 106 are next locked in the locking device 100 by inserting the anchor insert 200 into the anchor base 102. The excess sutures are then cut about the top surface of the anchor insert 200.

Exemplary Graft Fixation—Tibia:

1. Once the femur is fixed, the arthroscope is placed back in the knee and the graft is observed for tension and any sign of roof impingement as the knee is brought into full extension. Isometry can be evaluated by placing a hemostat on the graft sutures flush with the cannula, and re-tensioning can be achieved as necessary. The knee is placed in full hyperextension at this time.

2. A second anchor base 102 is then loaded with the graft sutures and advanced to the bone and impacted in place while keeping tension on the strands.

3. Finally the sutures 106 are locked in the locking device 100 by inserting the anchor insert 200 into the anchor base 102. The excess sutures are then cut about the upper surface of the anchor insert 200.

In at least some embodiments, a quadrupled semitendinosus graft ACL reconstruction process using the locking device to secure sutures at each of the femoral cortex and tibial cortex can be performed that includes the various steps:
a. harvest semitendinosus tendon graft;
b. prepare the graft by quadrupling around a soft, strong suture or tape material;
c. pretension the graft to remove deformable interfaces;
d. measure the graft diameter;
e. place outside-in pins in the femur and the tibia;
f. over-drill the guide pin;
g. impact retro-reamer or deploy fins, ream and retract and remove;
h. punch a cannula into the femoral and tibial cortices over the guide pin using introducer/guide—the cannula will be used to eventually tighten the graft in the knee;
i. suture passers (a suture shuttle with two openings) are placed through femoral and tibial cannulas and retrieved out the medial portal;
j. the sutures from the graft 108 are passed into the shuttle and pulled up through the medial portal and out the cannulas to allow the graft to be pulled into the knee, first into the femoral socket and then into the tibial socket;
k. tighten the graft in the knee by grasping sutures with a straight hemostat and twisting it against the back of the cannula;
l. remove the cannula;
m. on the femoral side, pass sutures 106 through a first anchor base 102 of a first locking device and impact the anchor base 102 in place in the bone 104;
n. a first anchor insert 200 is threaded with the sutures and inserted into the first anchor base 102 to lock the sutures 106 against the cleats 140;
o. arthroscopically check graft for impingement and graft tension;
p. in the same fashion as the femoral side fix sutures in device in tibia using a second locking device 100; and
q. cut the excess sutures protruding from the top of the anchor insert 200 on both the femur and tibia.

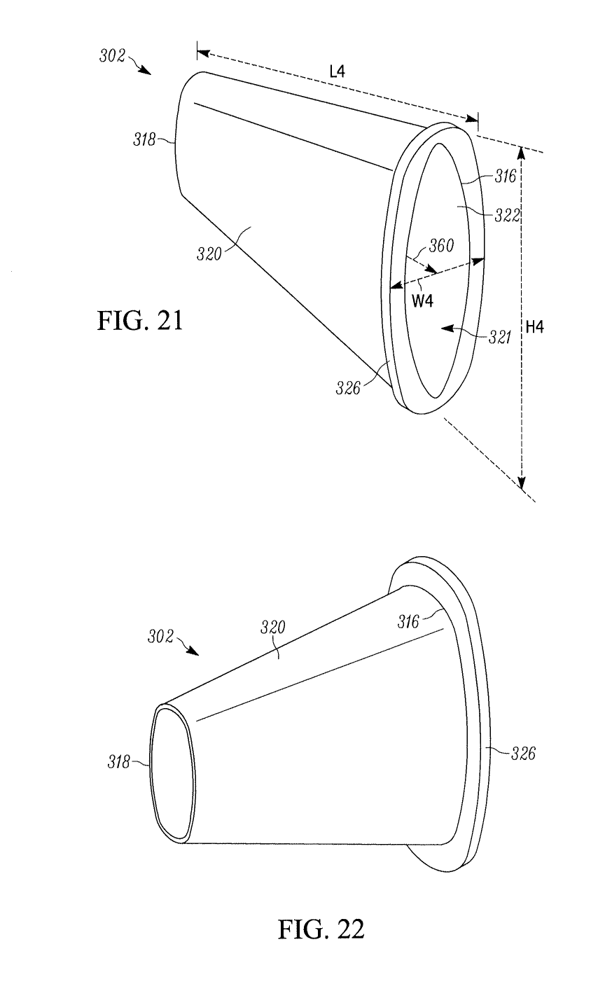

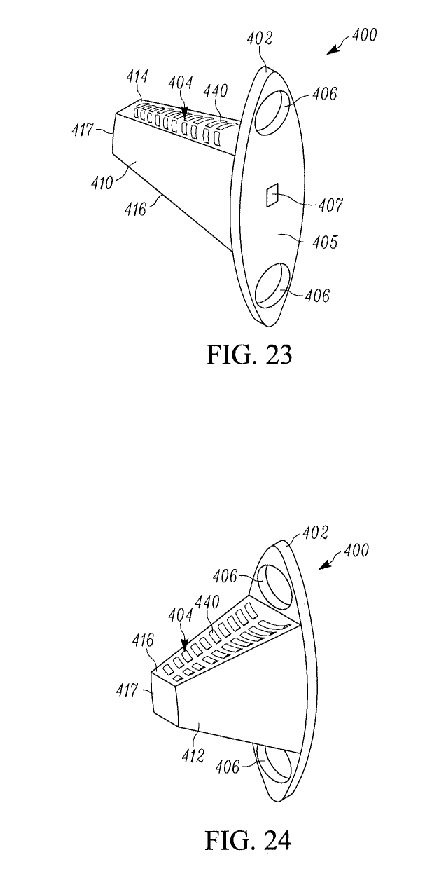

Referring now to FIGS. 21-24, another exemplary embodiment of a locking device 300 is provided. The locking device 300 includes an anchor base 302 (FIGS. 21 and 22) and an anchor insert 400 (FIGS. 23 and 24), with the anchor insert 400 configured for insertion into the anchor base 302, similar to anchor base 102 and anchor insert 200. The anchor base 302 includes similar elements to anchor base 102, such as a base inner cavity 321 formed by one or more base inner walls 322, an outer wall 320, a base bottom 318, a base top 316, and in at least some embodiments, a lip 326. For reference, the anchor base 302 includes a longitudinal center axis 360. The principal difference between anchor base 302 and anchor base 102 is that in at least some embodiments, the anchor base 302 lacks the cleats 140 protruding from the one or more base inner walls 122. Although in at least some embodiments, the anchor base 302 can include cleats on both the base inner walls 322 (similar to as shown in FIGS. 2A and 2B) and on the anchor insert 400 (FIGS. 22 and 23). Likewise, in at least some embodiments, the anchor base 102 can include cleats secured to or otherwise formed on both the one or more base inner walls 122 and on the anchor insert 200 (e.g., on the first insert side wall 214 and the second insert side wall 216, similar to FIGS. 22 and 23).

The anchor insert 400 includes similar elements to anchor insert 200, such as an insert top portion 402 and an insert wedge portion 404, wherein in at least some embodiments, the insert top portion 402 may or may not extend beyond the extents of the insert wedge portion 404 (similar to FIG. 11), and may or may not include apertures 406. Further, in at least some embodiments the insert wedge portion 404 includes a first insert outer wall 410 and an second insert outer wall 412, wherein the first insert outer wall 410 and second insert outer wall 412 are joined by a first insert side wall 414 and a second insert side wall 416. The anchor insert 400 further includes an insert bottom 417. The anchor insert 400 further includes cleats 440, with the cleats 440 secured to or otherwise protruding from at least one of the first insert side wall 414 and the second insert side wall 416 As with the anchor insert 200, anchor insert 400 can include more or less walls and the location of the cleats can vary. The cleats 440, in at least some embodiments, are similar to cleats 140, although in other embodiments, they can vary in size and shape as discussed above with regard to cleats 140. Further, as noted above, cleats can be provided on both the anchor base and the anchor insert, for example, the anchor base 201 can be used or configured for use with the anchor insert 400 by sizing the walls and cleats of each to allow insertion of the anchor insert 400 and to provide securement of the one or more sutures positioned inside the anchor base 201. It should be understood that the various sizes, shapes, dimensions, configurations, and embodiments discussed above with regard to the locking device 100 can be applicable to the locking device 300, again noting that the primary difference is the positioning of cleats on the anchor base and anchor insert. As such, the anchor base 302 includes a length L4, a width W4, and a height H4, which in at least some embodiments is the same as anchor base 102. Likewise, anchor insert 400 includes heights, length, and widths (similar to as shown in FIGS. 8B and 9 with reference to anchor insert 200) that can be identical or similar to the anchor insert 200 as described above.

The locking devices 100 and 300 are comprised of materials suitable for insertion into a living body, such as polyethylene, PEEK, carbon fiber, cobalt chrome, steel, aluminum, and/or other alloys, metals, ceramics, or polymer composites. The aforementioned material lists are not exclusive and therefore, other materials not listed or currently unknown can be used if suitable for such a purpose. Although the aforementioned procedure is described in relation to an ACL repair in a knee joint, the components and procedures can be modified to accommodate repair and/or replacement of various other ligaments in the knee (PCL) and other joints, such as a shoulder joint, as well as various other body parts that would benefit from suture securement in a living body. Further, the device and procedures can be modified to accommodate repair and/or replacement joints in the body of an animal as well as a human. In addition, all dimensions are exemplary and can vary as needed to accommodate a suitable procedure and patient.

Operation of the locking device 300 is similar to operation of locking device 100 in that the anchor base 302 is implanted in a bone with sutures passed through the base inner cavity 321 and then the anchor insert 400 is then inserted into the anchor base 302 to secure the sutures therein. It is to be understood that the installation of the anchor insert 302 into the bone can be accomplished similar to the implantation of the locking device 100 as discussed above.

It should be appreciated that the present disclosure is intended to encompass numerous embodiments as disclosed herein and further described by the following:
(i). A locking device comprising:
   an anchor base having a base outer wall and one or more base inner walls, wherein the one or more base inner walls form a base inner cavity;
   one or more cleats extending from the one or more base inner walls;
   an anchor insert having a first insert outer wall and a second insert outer wall, wherein the anchor insert is shaped to be received within the anchor base to provide securement of one or more sutures to the cleats, when sutures are situated inside the base inner cavity.
(ii). The locking device of (i), wherein the anchor insert is tapered as it extends from an insert top portion towards an insert bottom, and wherein the base inner cavity is tapered as it extends from a base top towards a base bottom.
(iii). The locking device of any one of (i)-(ii), wherein the one or more base inner walls include a first base inner wall joined to a second base inner wall by a first base corner and a second base corner.
(iv). The locking device of any one of (i)-(iii), wherein the first base corner and the second base corner are formed as wedge shapes.
(v). The locking device of any one of (i)-(iv), wherein the one or more cleats includes a column of one or more first cleats extending at least partially along the length of the first base corner, and a column of one or more second cleats extending at least partially along the length of the second base corner, and wherein the first base corner in combination with the first cleats forms a first grip portion and the second base corner in combination with the second cleats forms a second grip portion.
(vi). The locking device of any one of (i)-(v), wherein the one or more cleats includes a column of first cleats situated opposite a column of second cleats, with the first and second cleats extending at least partially along the length of the first base corner, and a column of third cleats situated opposite a column of fourth cleats extending at least partially along the length of the second base corner, and wherein the first base corner in combination with the first and second cleats forms a first grip portion and the second base corner in combination with the third and fourth cleats forms a second grip portion.
(vii). The locking device of any one of (i)-(vi), wherein the first cleats and second cleats are angled with respect to a central longitudinal axis of the anchor base.
(viii). The locking device of any one of (i)-(vii), wherein the first grip portion engages with a first insert side wall of the anchor insert and the second grip portion engages with a second insert side wall of the anchor insert when the anchor insert is received into the base inner cavity.
(ix). The locking device of any one of (i)-(viii), wherein the base bottom includes an aperture for receiving one or more sutures therethrough and the anchor base includes at least one of a first grip portion and a second grip portion for receiving the one or more sutures.
(x). The locking device of any one of (i)-(ix), wherein the insert top portion includes one or more apertures and the base bottom includes an aperture for receiving one or more sutures therethrough.
(xi). The locking device of any one of (i)-(x), wherein the anchor insert is securable to the anchor base via friction only.
(xii). The locking device of any one of (i)-(xi), wherein the anchor insert is securable to the anchor base via a locking assembly that includes a locking protrusion extending from one of the anchor insert and the anchor base that is engageable with a slot for receiving the locking protrusion in the other of the anchor insert and the anchor base.
(xiii). The locking device of any one of (i)-(xii), wherein the base top includes an outer lip that protrudes radially from the base outer wall.
(xiv). The locking device of any one of (i)-(xiii), wherein the base outer wall is textured to provide frictional engagement with at least one of an aperture wall formed in a bone or an adhesive applied to the aperture wall formed in the bone.
(xv). The locking device of any one of (i)-(xiv), wherein the first grip portion receives and secures a first end of a suture and the second grip portion receives and secures a second end of the same suture, and wherein the anchor base and anchor insert are comprised of material suitable for insertion in a living body.
(xvi). A locking device comprising:
   a base having a base outer wall and an inner cavity;
   a first base inner wall joined to a second base inner wall at a first base corner and a second base corner to form the inner cavity, wherein the first base corner includes a first grip portion having one or more cleats, and the second base corner includes a second grip portion having one or more cleats;
   an insert having a first insert outer wall and a second insert outer wall, wherein the first insert outer wall and second insert outer wall are joined by a first insert side wall and a second insert side wall; and wherein the anchor insert is shaped to be received at least partially within the base.
(xvii). The locking device of (xvi), wherein the insert includes an insert wedge portion that tapers down as it extends from an insert wedge top towards an insert bottom, and wherein the inner cavity of the base is tapered as it extends from a base top towards a base bottom.
(xviii). The locking device of any one of (i)-(xvii), wherein the insert in cooperation with the base provides securement of one or more sutures to one or both of the first grip portion and the second grip portion.
(xix). The locking device of any one of (i)-(xviii), wherein the first grip portion and second grip portion are configured to engage with the first insert side wall and the second insert side wall, respectively, to receive and secure one or more sutures.
(xx). The locking device of any one of (i)-(xix), wherein the base is not a screw.
(xxi). The locking device of any one of (i)-(xx), wherein the base does not include threads on the base outer wall.
(xxii). The locking device of any one of (i)-(xxi), wherein the base outer wall is not configured to engage one or more sutures.
(xxvii). The locking device of any one of (i)-(xxii), wherein the base is insertable into a bone via impact only.
(xxiv). The locking device of any one of (i)-(xxvii), wherein the base cannot be threaded into a bone.
(xxvi). A locking device comprising:
   an anchor base having a base outer wall and one or more base inner walls, wherein the one or more base inner walls form a base inner cavity;
   a base bottom and a base top, wherein the base bottom and base top each include an opening therethrough;
   an anchor insert having a first insert outer wall and a second insert outer wall, wherein the anchor insert is shaped to be received within the anchor base;
   one or more cleats extending from at least one of the one or more base inner walls, the first insert outer wall, and the second insert outer wall.
(xxvi). The locking device of (xxvi) in combination with any one of (ii)-(xv).
(xxvii). A ligament reconstruction process comprising:
   a. securing a first suture to a first end of a tendon graft;
   b. securing a second suture to a second end of the tendon graft;
   c. implanting a first anchor base at least partially within an opening in a first bone, wherein the first anchor base includes one or more cleats;
   d. implanting a second anchor base at least partially within an opening in a second bone, wherein the first anchor base includes one or more cleats, and wherein the first and second bones are associated with a joint;
   e. passing the first suture through the opening in the first bone and the first anchor base;
   f. passing the second suture through the opening in the second bone and the second anchor base;
   g. inserting a first anchor insert into the first anchor base to abut the first suture into engagement with the respective one or more cleats to secure the first suture within the first anchor base; and
   h. inserting a second anchor insert into the second anchor base to abut the second suture into engagement with the respective one or more cleats to secure the second suture within the second anchor base.
(xxviii). A ligament reconstruction process comprising:
   a. securing a first suture to a first end of a tendon graft;
   b. securing a second suture to a second end of the tendon graft;
   c. implanting a first anchor base at least partially within an opening in a first bone;
   d. implanting a second anchor base at least partially within an opening in a second bone, and wherein the first and second bones are associated with a joint;
   e. passing the first suture through the opening in the first bone and the first anchor base;
   f. passing the second suture through the opening in the second bone and the second anchor base;
   g. inserting a first anchor insert into the first anchor base to abut the first suture into engagement with one or more cleats on one of the first anchor base and the first anchor insert to secure the first suture within the first anchor base; and
   h. inserting a second anchor insert into the second anchor base to abut the second suture into engagement with one or more cleats on one of the second anchor base and the second anchor insert to secure the second suture within the second anchor base.

It is specifically intended that the locking device and method of use not be limited to the embodiments and illustrations contained herein, but include modified forms of those embodiments including portions of the embodiments and combinations of elements of different embodiments as come within the scope of the following claims. Further, the steps outlined above can be modified in various manners, such as performance in one or more alternate orders. The addition or exclusion of any step(s) discussed or not discussed, does not preclude a desired completion of the procedure. Numerical ranges disclosed herein include all values from, and including, the lower value and the upper value.

We claim:

1. A locking device comprising:
an anchor base having a base outer wall and one or more base inner walls, wherein the one or more base inner walls form a base inner cavity;
one or more cleats extending from the one or more base inner walls;
an anchor insert having a first insert outer wall and a second insert outer wall, wherein the anchor insert is shaped to be received within the anchor base to provide securement of one or more sutures to the cleats, when sutures are situated inside the base inner cavity.

2. The locking device of claim 1, wherein the anchor insert is tapered as it extends from an insert top portion towards an insert bottom, and wherein the base inner cavity is tapered as it extends from a base top towards a base bottom.

3. The locking device of claim 2, wherein the one or more base inner walls include a first base inner wall joined to a second base inner wall by a first base corner and a second base corner.

4. The locking device of claim 3, wherein the first base corner and the second base corner are formed as wedge shapes.

5. The locking device of claim 3, wherein the one or more cleats includes a column of one or more first cleats extending at least partially along the length of the first base corner, and a column of one or more second cleats extending at least partially along the length of the second base corner, and wherein the first base corner in combination with the first cleats forms a first grip portion and the second base corner in combination with the second cleats forms a second grip portion.

6. The locking device of claim 3, wherein the one or more cleats includes a column of first cleats situated opposite a column of second cleats, with the first and second cleats extending at least partially along the length of the first base corner, and a column of third cleats situated opposite a column of fourth cleats extending at least partially along the length of the second base corner, and wherein the first base corner in combination with the first and second cleats forms a first grip portion and the second base corner in combination with the third and fourth cleats forms a second grip portion.

7. The locking device of claim 6, wherein the first cleats and second cleats are angled with respect to a central longitudinal axis of the anchor base.

8. The locking device of claim 6, wherein the first grip portion engages with a first insert side wall of the anchor insert and the second grip portion engages with a second insert side wall of the anchor insert when the anchor insert is received into the base inner cavity.

9. The locking device of claim 2, wherein the base bottom includes an aperture for receiving one or more sutures therethrough and the anchor base includes at least one of a first grip portion and a second grip portion for receiving the one or more sutures.

10. The locking device of claim 2, wherein the insert top portion includes one or more apertures and the base bottom includes an aperture for receiving one or more sutures therethrough.

11. The locking device of claim 10, wherein the anchor insert is securable to the anchor base via friction only.

12. The locking device of claim 10, wherein the anchor insert is securable to the anchor base via a locking assembly that includes a locking protrusion extending from one of the anchor insert and the anchor base that is engageable with a slot for receiving the locking protrusion in the other of the anchor insert and the anchor base.

13. The locking device of claim 9, wherein the base top includes an outer lip that protrudes radially from the base outer wall.

14. The locking device of claim 13, wherein the base outer wall is textured to provide frictional engagement with at least one of an aperture wall formed in a bone or an adhesive applied to the aperture wall formed in the bone.

15. The locking device of claim 9, wherein the first grip portion receives and secures a first end of a suture and the second grip portion receives and secures a second end of the same suture, and wherein the anchor base and anchor insert are comprised of material suitable for insertion in a living body.

16. A locking device comprising:
a base having a base outer wall and an inner cavity;
a first base inner wall joined to a second base inner wall at a first base corner and a second base corner to form the inner cavity, wherein the first base corner includes a first grip portion having one or more cleats, and the second base corner includes a second grip portion having one or more cleats;
an insert having a first insert outer wall and a second insert outer wall, wherein the first insert outer wall and second insert outer wall are joined by a first insert side wall and a second insert side wall; and wherein the anchor insert is shaped to be received at least partially within the base.

17. The locking device of claim 16, wherein the insert includes an insert wedge portion that tapers down as it extends from an insert wedge top towards an insert bottom, and wherein the inner cavity of the base is tapered as it extends from a base top towards a base bottom.

18. The locking device of claim 16, wherein the insert in cooperation with the base provides securement of one or more sutures to one or both of the first grip portion and the second grip portion.

19. The locking device of claim 16, wherein the first grip portion and second grip portion are configured to engage with the first insert side wall and the second insert side wall, respectively, to receive and secure one or more sutures.

20. A ligament reconstruction process comprising:
a. securing a first suture to a first end of a tendon graft;
b. securing a second suture to a second end of the tendon graft;
c. implanting a first anchor base at least partially within an opening in a first bone;
d. implanting a second anchor base at least partially within an opening in a second bone, and wherein the first and second bones are associated with a joint;
e. passing the first suture through the opening in the first bone and the first anchor base;
f. passing the second suture through the opening in the second bone and the second anchor base;
g. inserting a first anchor insert into the first anchor base to abut the first suture into engagement with one or more cleats on one of the first anchor base and the first anchor insert to secure the first suture within the first anchor base; and
h. inserting a second anchor insert into the second anchor base to abut the second suture into engagement with one or more cleats on one of the second anchor base and the second anchor insert to secure the second suture within the second anchor base.

21. A locking device comprising:
an anchor base having a base outer wall and one or more base inner walls, wherein the one or more base inner walls form a base inner cavity;
a base bottom and a base top, wherein the base bottom and base top each include an opening therethrough;
an anchor insert having a first insert outer wall and a second insert outer wall, wherein the anchor insert is shaped to be received within the anchor base;
one or more cleats extending from at least one of the one or more base inner walls, the first insert outer wall, and the second insert outer wall.
